(19) United States
(12) Patent Application Publication
(10) Pub. No.: US 2022/0280441 A1
(43) Pub. Date: Sep. 8, 2022

(54) FILM WITH LATERALLY ADJOINED STRIPS FOR ADMINISTRATION OF A VACCINE

(71) Applicant: CA Pharma Inc., Burlington (CA)

(72) Inventor: Michael Bousfield, Burlington (CA)

(21) Appl. No.: 17/752,325

(22) Filed: May 24, 2022

Related U.S. Application Data
(63) Continuation-in-part of application No. PCT/CA2021/051118, filed on Aug. 13, 2021.
(60) Provisional application No. 63/065,828, filed on Aug. 14, 2020.

(51) Int. Cl. A61K 9/70; A61K 9/00; A61K 39/13; A61K 47/26; A61K 47/36; A61K 47/38

(52) U.S. Cl. CPC A61K 9/7007; A61K 9/0056; A61K 39/13; A61K 47/26; A61K 47/36; A61K 47/38; A61K 2039/542

(57) ABSTRACT

A film for administration to a mucosal membrane is provided. The film comprises a first film strip comprising a vaccine in a first film matrix comprising at least one film-forming agent, and at least one of a surfactant, emulsifier and/or a plasticizer, and a second film strip comprising an adjuvant in a second film matrix comprising at least one film-forming agent, and at least one of a surfactant, emulsifier and/or a plasticizer. The first and second strips of the film are laterally adjoined along an edge of the first and second strips to provide a single layer film in which the vaccine and adjuvant are not admixed prior to administration.

### FIELD OF THE INVENTION

The invention generally relates to thin film technology, and more specifically, to films useful for mucosal delivery of a therapeutic agent such as a vaccine.

### BACKGROUND OF THE INVENTION

Orally disintegrating or dissolving edible film-based materials are currently used in a variety of embodiments for conveniently holding and delivering nutrients, flavors and medicinal compounds such as breath fresheners and vitamins. For example, sublingual oral care strips that provide a breath-freshening function have been in existence for some time, and typically comprise a film-forming agent and a flavor agent such as menthol, peppermint or spearmint, as described in U.S. Pat. No. 6,596,298.

Novel forms of consumable edible thin films have more recently been developed as described, for example, in U.S. Patent Application No. 2004/0096569 to Barkalowetal that vary in color, flavor, shape and mouthfeel, to improve the marketability of edible films to consumers. U.S. 2003/0224090 describes edible films as snacks devised to provide flavor and/or craving satisfaction. These films comprise high levels of appealing flavors and flavor combinations, as well as sweeteners and other ingredients. The snack may include one or more layers to preserve structure, or to enhance flavor sensation.

The use of edible films for the delivery of therapeutics is an attractive alternative to other delivery forms, including delivery by other oral dosage forms. Oral vaccination, for example, is appealing for use with populations that are needle-adverse (e.g. children), and is beneficial due to the ease of regulatory compliance and reduced cost. Oral immunization has been used in vaccine campaigns against influenza, poliomyelitis, rotavirus, typhoid fever, cholera, and other diseases. However, only a few vaccines have been effectively formulated for oral administration, since there are issues with oral administration that may reduce efficacy. For example, to be effective, the antigen must remain intact until it reaches the lumen of the intestine without degradation by the acidic environment of the stomach. Otherwise, the antigen concentration may diminish from levels capable of inducing an immune response to an anergy response. In addition to these challenges, relatively little progress has been made in the manufacturing an effective formulation comprising an adjuvant to enhance the immunogenic effect of the vaccine.

It would be desirable, thus, to develop a film for mucosal delivery of a vaccine.

### SUMMARY OF THE INVENTION

Novel films are herein provided comprising laterally adjoined film strips for the effective mucosal delivery of a vaccine in combination with an adjuvant.

Thus, in one aspect of the invention, a film is provided comprising a first strip comprising a vaccine and at least one film-forming agent, and optionally, a surfactant, an emulsifier and/or a plasticizer, and a second strip comprising an adjuvant and at least one film-forming agent, and optionally, a surfactant, emulsifier and/or a plasticizer, wherein said first and second strips are laterally adjoined at an edge of the first and second strips.

In another aspect of the invention, a method of vaccination is provided comprising administering to a patient, a film comprising two laterally adjoined strips as above described, wherein the first strip comprises a vaccine and the second strip comprises an adjuvant.

This and other aspects of the invention are described by reference to the following figures which exemplify embodiments of an aspect of the invention.

### BRIEF DESCRIPTION OF THE FIGURES

FIG. 1 illustrates a single layer film dose comprising laterally adjoined strips with two matrices, each comprising 50% of the film dose;

FIG. 2 illustrates a single layer film dose comprising laterally adjoined strips with two different matrices, one comprising ⅓ of the film dose and the other comprising ⅔ of the film dose;

FIG. 3 illustrates a single layer film dose comprising laterally adjoined strips with two different matrices, one comprising ¼ of the film dose and the other comprising ¾ of the film dose;

FIG. 4 illustrates a single layer film comprising laterally adjoined strips with two different matrices, one comprising ⅙ of the film dose and the other comprising ⅚of the film dose;

FIG. 5 illustrates a single layer film comprising laterally adjoined strips with 3 film matrices, each comprising ⅓ of the film dose;

FIG. 6 illustrates a single layer film comprising laterally adjoined strips with 3 film matrices, two each comprising 25% of the film dose, and the third comprising 50% of the film dose;

FIG. 7 illustrates a single layer film comprising laterally adjoined strips with 4 film matrices, each comprising ¼ of the film dose; and

FIG. 8 illustrates a single layer film comprising laterally adjoined strips with 6 film matrices, each comprising ⅙ of the film dose.

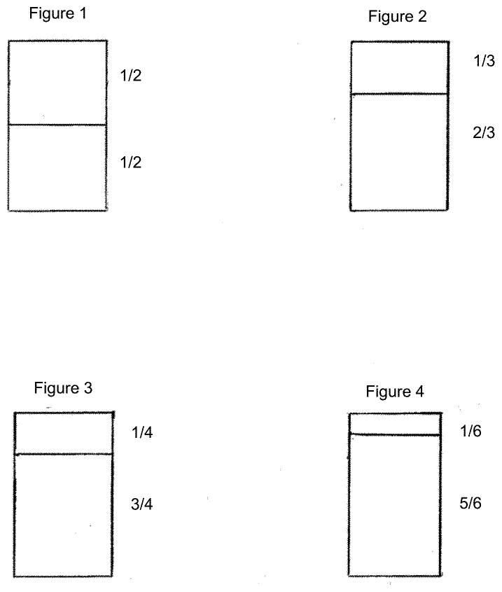

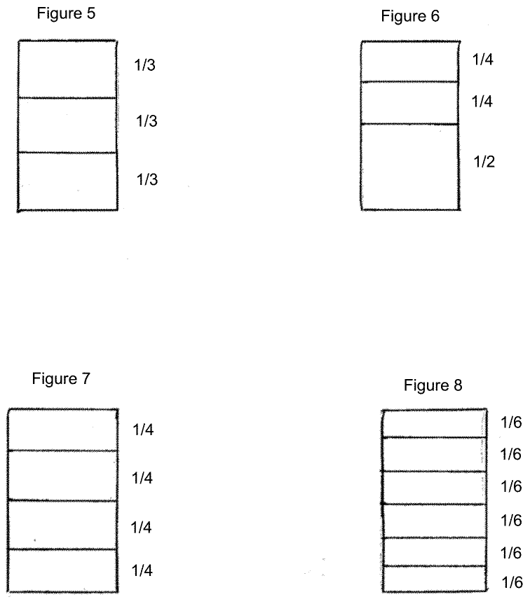

### DETAILED DESCRIPTION OF THE INVENTION

A single layer film is provided comprising a first film strip comprising a vaccine in a first film matrix comprising at least one film-forming agent, and a second film strip comprising an adjuvant in a second film matrix comprising at least one film-forming agent, wherein the first and second film matrices optionally comprise a surfactant, an emulsifier and/or a plasticizer, wherein said first and second strips are laterally adjoined along an edge of the first and second strips.

The phrase “laterally adjoined” as it relates to the first and second strips of the edible film refers to strips which are joined together along an edge of each strip, in a side-by-side fashion, to form a single layer film comprising two or more strips in which the strips are oriented edge to edge, as opposed to face to face, and thus, form a continuous plane, i.e. lie along the same plane. The present film comprises a single film layer as opposed to a layered film in which film strips are laid face to face, one on top of another, as in a laminate. The term “edge” as used herein with respect to a film strip refers to the thinnest side of each film strip. The edge of a strip coincides with the thickness of the strip. A “face” of a film strip is the surface of the strip comprising the largest area of the film strip.

The matrix of each film strip of the present film product comprises at least one physiologically acceptable film forming agent. The term “physiologically acceptable” is used herein to refer to film-forming agents that are acceptable for administration to, or use in, a mammal and that exhibit minimal or no adverse side effects on use. Suitable film-forming agents for use in the present film product include, but are not limited to, pullulan, cellulose-based agents such as methyl cellulose, ethyl cellulose, hydroxypropylmethyl cellulose, hydroxymethyl cellulose, hydroxyethyl cellulose and hydroxypropyl cellulose, algin-based agents such as sodium alginate, carrageenan, amylose, high amylose starch, hydroxypropylated high amylose starch, corn starch, cassava starch, glucan, dextrin, pectin, chitin, chitosan, collagen, gelatin, zein, levan, elsinan and mixtures thereof.

The film forming agent(s) may be used alone or combined with one or more secondary film forming agents that facilitate film forming, including but not limited to, one or more of polyethylene glycol, polypropylene glycol, polylactic acid, sodium methacrylic acid polymers, methacrylic acid copolymers, acrylic acid polymers, acrylic acid copolymers, carboxyvinyl polymer, polyvinylpyrrolidone (PVP), carbopol, polycarbophil, cellulose gum, xanthan gum, tragacanth gum, guar gum, acacia gum, arabic gum, karaya gum, welan gum, locust bean gum, gellan gum, casein, maize, chondruscrispus, crosspovidone (CPV), glycogen, phytoglycogen, polysaccharides, hydroxypropylate, dextrin, maltodextrin, soy protein isolate, whey protein isolate, beeswax, camauba wax, fungal exopolysaccharides, rapeseed, and mixtures thereof.

The film forming agent(s), or combination of film-forming agent(s) and secondary film forming agent(s), are present in a combined amount ranging from about 0.01 to about 99 wt %, preferably from about 10 to about 80 wt %, more preferably from about 15 to about 75 wt % of the present film product, and even more preferably, from about 20 to about 60 wt % of the film, e.g. 25% by wt. Preferred film forming agents include pullulan, algin-based agents and cellulose-based agents, e.g. pullulan, sodium alginate and hydroxypropyl methylcellulose.

The film matrices within each film strip may also include a surfactant, i.e. a compound that lowers the surface tension (or interfacial tension) between two liquids or between a liquid and a solid. Examples of surfactants include, but not limited to, glycerol, glycerin, propylene glycol, sucrose fatty acid ester, sorbitan monostearate, sorbitol-based surfactants such as polysorbate 60 or 80, lecithin such as soy or sunflower lecithin, modified cellulose gum (CMC), ceratonia gum (locust bean gum), alkali metal alkyl sulfates such as sodium lauryl sulfate (SLS) and sodium dodecyl sulfate, or mixtures thereof. The film will generally comprise a surfactant in an amount ranging from about 0.5 to 15% by wt, e.g. 0.5 to 12.7 wt %.

An emulsifier is a type of surfactant, and functions to stabilize an emulsion. The film matrices may include an emulsifier, preferably in an amount in the range of about 1% to 15% by wt. Examples of suitable emulsifiers include but are not limited to polyoxyethylene, sorbitan monostearate, lecithin such as soy lecithin, stearate salt such as potassium stearate, calcium stearate or sodium stearate, calcium stearoyl lactylate (CSL), glyceryl monostearate, mono propylene glycol, sorbitan monooleate (SPAN 80), sorbitol-based emulsifiers such as Polysorbate 20, 40, 60 or 80 (Tween 60/80), monoglyceride, glyceroltriacetate and mixtures thereof.

The film matrices may include a surfactant or an emulsifier, or may include a surfactant as well as an emulsifier, for example, sorbitol-based surfactants, such as polysorbate 20, 40, 60 or 80, glycerin or propylene glycol, combined with emulsifiers such as sorbitan monostearate, a stearate salt, or polyoxyethylene.

The film matrices may also include plasticizer that increases the plasticity and/or decreases the viscosity of the film. Examples of plasticizers include, but are not limited to, monostearate palm oil, lethicin, sorbitan monostearate, glycerin, glycerin fatty acid ester, triacetin, pre-gelatinized starch, sorbitol, medium chain mono- or diglycerides, castor oil, malic acid, polyethylene glycol (PEG), glycerol, palm oil and mixtures thereof. The film will generally comprise plasticizer in an amount ranging from about 2 to 18 wt %.

It is noted, as one of skill in the art will appreciate, that some compounds may have multiple functions, and thus, satisfy multiple roles in the present film matrices, for example, sorbitol-based compounds function as both a surfactant and an emulsifier, sorbitan monostearate and lecithin function as a surfactant, emulsifier and plasticizer, and glycerol functions as both a surfactant and a plasticizer. Thus, the presence of one compound may satisfy more than one function in the present film.

As will be appreciated by one of skill in the art, each film strip may comprise the same or different film-forming components. The provision of laterally adjoined strips within the film allows for this flexibility in the composition of the strips, and permits each strip to be optimized based on the active ingredient or other components to be incorporated into each strip for delivery, i.e. the vaccine or adjuvant, or other active ingredient that a strip comprises. Thus, each strip may be varied in composition to provide strips with different dissolution properties, delivery properties, adhesion properties, flavours, colour, or other properties, as well as combinations of these properties, as desired or required.

In one embodiment, the present film may comprise one or more fast-dissolving laterally adjoined film strips in which the film forming agent is selected from pullulan, sodium alginate, and mixtures thereof to result in a film that dissolves, for example, in 1-60 seconds as measured using a dissolve rate test such as a permeation test that mimics the oral environment. In an example, quick dissolving film strips comprise pullulan in an amount ranging from 1 to 99%, optionally combined with sodium alginate, in an amount ranging from about 1 to 99%. The strips may exhibit different dissolution rates by altering the film forming agent content of the strips. For example, by using a fast dissolving film forming agent, or a greater content of a fast dissolving film forming agent such as pullulan, the dissolution rate of the film can be increased, while using a film forming agent that dissolves at a slower rate, e.g. alginate, or a greater content of such a film forming agent, the dissolution rate of the film is decreased. Dissolution may also be altered by adjusting the pH of the film, for example, increasing the pH of the film, will generally increase dissolution rate of the film.

In another embodiment, the present film may comprise one or more slow dissolving laterally adjoined strips in which the film forming agent is selected from hydroxypropylmethylcellulose (HPMC), carrageen, and mixtures thereof, additionally comprising one or more facilitating agents such as maltodextrin, to result in a film that dissolves, for example, in 61-180 seconds according to a permeation test. By altering the film forming agent content of a strip, different dissolution rates may be achieved. For example, a slow dissolving film may be attained which comprises hydroxypropyl methylcellulose, carrageen and maltodextrin in amounts ranging from 11-45% by wt HPMC, 1-23% by wt carrageen and 5-29% by wt maltodextrin. Higher amounts of HPMC would result in a slower dissolving strip, while higher amounts of sodium alginate would result in a strip which dissolves less slowly.

A film may be provided comprising one or more fast-dissolving laterally adjoined strips, as well as one or more slow dissolving strips. Thus, one strip may comprise pullulan, sodium alginate, or mixtures thereof to form a fast-dissolving strip and another strip may comprise hydroxypropylmethylcellulose, carrageen, or mixtures thereof to form a slower-dissolving strip or a strip that dissolves at a reduced rate. The provision of a film comprising strips that dissolve at different rates permits staged dissolution and delivery of the film's contents.

Any suitable method may be used to determine dissolve rate of a film. An exemplary method includes tautly fastening a sample of the film (preferably, 30 to 35 mm long, 20-25 mm in width and a thickness of 0.053 mm-0.063 mm) with adhesive tape on the raised test plate of a dissolution testing apparatus. A chrome ball bearing (about 6.35 mm in diameter, and 1.045+/−0.002 g) is placed centrally on top of the film and 2 drops of potable water at ambient temperature are dropped onto the top of the ball bearing. The time for the ball permeates the film sample and drop to the lower test plate 10 mm below represents the dissolution rate. The longer the time, the slower the dissolution rate. This method is referred to herein as EI QC019.

Films according to the present invention which include film strips of the same composition may also be prepared which maintain the benefit of the laterally adjoined strips. An advantage of providing a film with laterally adjoined strips is the ability to prepare each strip separately so as not to prematurely combine active ingredients, i.e. prematurely combine the vaccine and adjuvant, which may result in reduced immunogenic effect of the vaccine. For example, for vaccines which comprise a peptide antigen rather than whole virus, adsorption of peptide antigens onto the surface of metallic adjuvants may result in partial unfolding of their conformational structure, rendering them either less immunogenic or completely inert. This process may be magnified at the elevated manufacturing temperatures necessary to produce films such as the film of the present invention. Thus, provision of a film in which the vaccine is separated from the adjuvant to prevent contact prior to administration of the film as described is beneficial to maintain optimal efficacy of the vaccine.

The present film comprises within a first film strip a vaccine combined with a first film matrix. The present film is not restricted with respect to the type of vaccine that can be incorporated within the first film strip of the present film. Accordingly, suitable vaccines for incorporation with the first film strip include, live-attenuated vaccines such as vaccines typically used to immunize against Measles, Mumps and Rubella (MMR combined vaccine), Rotavirus, Smallpox, Chickenpox and Yellow fever; inactivated vaccines such as vaccines used to immunize against Hepatitis A, Influenza, Polio and Rabies; subunit, recombinant, polysaccharide, or conjugate vaccines comprising a portion of the target virus, e.g. protein, sugar or capsid, such as vaccines used to immunize against Hepatitis B, Human papillomavirus, Shingles, Pneumococcal disease and meningococcal disease; or toxoid vaccines based on a toxin produced by the target virus such as the vaccine used to immunize against Diptheria or Tetanus.

The vaccine for use in the film may target a virus from any of a multitude of virus families, including both DNA viruses and RNA viruses, for example, Adenoviridae (Adenovirus, infectious canine hepatitis virus), Alphasatellitidae, Ampullaviridae, Anelloviridae (Torque teno virus), Ascoviridae, Asfarviridae, Bacilladnaviridae,Baculoviridae, Bicaudaviridae, Bidnaviridae, Caudovirales, Circoviridae, Clavaviridae, Corticoviridae, Fuselloviridae, Geminiviridae, Genomoviridae, Globuloviridae, Guttaviridae, Hepadnaviridae, Herpesvirales (Herpes simplex virus, varicella-zoster virus, cytomegalovirus, Epstein-Barr virus), Hytrosaviridae, Inoviridae, Iridoviridae, Lavidaviridae,Ligamenvirales, Marseilleviridae, Micro viridae, Mimiviridae, Nanoviridae, Nimaviridae, Nudiviridae, Ortervirales, Ovaliviridae, Papillomaviridae (Papillomavirus, polyomaviridae, simian vacuolating virus), Parvoviridae (Parvovirus B19, canineparvovirus), Phycodnaviridae, Plasmaviridae, Pleolipoviridae (HHPV1, HRPV1, HGPV1, His2V), Polydnaviridae, Polyomaviridae, Portogloboviridae, Poxviridae (Smallpox virus, cow pox virus, sheep pox virus, orf virus, monkey pox virus, vaccinia virus), Smacoviridae, Sphaerolipoviridae, Spiraviridae, Tectiviridae, Tolecusatellitidae, Tristromaviridae, Turriviridae, Reoviridae (Reovirus, rotavirus), Picornaviridae (Enterovirus, rhinovirus, hepatovirus, cardiovirus, aphthovirus, poliovirus, parechovirus, erbovirus, kobuvirus, teschovirus, coxsackie) , Caliciviridae (Norwalk virus) , Togaviridae (Rubella virus, Eastern equine encephalitis) , Arenaviridae (Lymphocytic choriomeningitis virus, Lassa fever) , Flaviviridae (Dengue virus, hepatitis C virus, yellow fever virus, Zika virus) , Orthomyxoviridae (Influenza virus A, Influenzavirus B, Influenza virus C, Isavirus, thogotovirus), Paramyxoviridae (Measles virus, mumps virus, respiratory syncytial virus, Rinderpest virus, canine distemper virus), Bunyaviridae (California encephalitis virus, Sin nombre virus) , Rhabdoviridae (Rabies virus, Vesicular stomatitis), Filoviridae (Ebola virus, Marburg virus), Coronaviridae (SARS-CoV-2, COVID-19), Astroviridae (Astrovirus), Bornaviridae (Borna disease virus) , Arteriviridae (Arterivirus, equine arteritis virus) and Hepeviridae (Hepatitis E virus).

Examples of vaccines that may be incorporated within the first strip of the present film include, but are not limited to, Actacel, ActHIB, COMVAX, H. influenzae D15 protein vaccine, H. influenzae Hap protein vaccine, H. influenzae Omp26 protein vaccine, H. influenzae Type b Capsular Polysaccharide Vaccine, Hiberix, Hibtiter, Infanrix-IPV/Hib, Infanrix-hexa, Infanrix/Hib, Killed nontypeable H. influenzae whole-cell vaccine, MenHibrix, Non-typeable H. influenzae dLOS-P6 vaccine, Non-typeable H. influenzae rLP4/rLP6 and Moraxella catarrhalis UspA2 protein vaccine, Nontypeable H. influenzae cell membrane (CM-Hi) vaccine, Nontypeable H. influenzae Hap Protein Vaccine, Nontypeable H. influenzae LOS-TT conjugate vaccine, Nontypeable H. influenzae NucA Protein vaccine, Nontypeable H. influenzae Outer Membrane Protein P1 vaccine, Nontypeable H. influenzae outer membrane recombinant P4 vaccine, nontypeable H. influenzae P5 peptide MVF/H3 vaccine, Nontypeable H. influenzae protein P6 with cholera toxin, Nontypeable H. influenzae rTbpB vaccine, Pediacel, PedvaxHIB, Pentacel, ProHIBiT and recombinant nontypeable H. influenzae protein P6 with AdDP.

The amount of vaccine incorporated within the first strip will depend on the particular vaccine and the disease to be treated. Generally, the amount of vaccine to be incorporated in a film strip is in the range of about 0.001% to 30% by wt of the film. An advantage of the present films is that they can incorporate a high load of vaccine, if required, e.g. a load in the range of about 10% to 30% by wt of the film.

The film also comprises a second film strip comprising an adjuvant and a second film matrix. The term “adjuvant” or “immunologic adjuvant” is used herein to refer to a substance that acts to accelerate, prolong, or enhance antigen-specific immune responses when used in combination with a vaccine. A suitable adjuvant may include a compound that functions in one or more of the following ways to enhance the immune response of a given vaccine:
1) the adjuvant may assist in the translocation of the vaccine to lymph nodes to be recognized by T cells, thereby increasing T cell activity to result in an increased clearance of the target virus;
2) the adjuvant may physically protect the vaccine to prolong its presence and effect. The longer the vaccine is present, the greater the production of B and T cells, and the greater the immunological memory created in a treated organism;
3) the adjuvant may induce a greater release of chemokines by helper T cells and mast cells;
4) the adjuvant may induce the release of inflammatory cytokines which help to recruit B and T cells at sites of infection and to increase transcriptional events leading to a net increase of immune cells as a whole; and

5) the adjuvant may increase the innate immune response to antigen by interacting with pattern recognition receptors (PRRs) on or within accessory cells.

In one embodiment, the adjuvant may be an inorganic entity, for example, a mineral-based adjuvant such as an aluminum-based adjuvant, e.g. aluminum hydroxide, aluminum phosphate, aluminum oxide or aluminum potassium sulfate, or calcium phosphate.

In another embodiment, the adjuvant is an organic entity. Examples of organic adjuvant include oils such as mineral oil (e.g. Freund's incomplete adjuvant of Freund's complete adjuvant (CFA)), paraffin oil, squalene and emulsions such as water-in-oil emulsions with emulsifiers such as mannide mono-oleate (e.g. Montanide ISA 720); saponins such as water-soluble triterpene glycosides (e.g. ISCOMS and ISCOMATRIX); cytokines such as IL-1 and IL-12; tensioactive agents or surfactants such as polyoxyethylene sorbitan fatty acid esters (Tweens), polyoxyethylene stearates, sorbitan fatty acid esters (Spans), ethylene or propylene oxide, sorbitan esters, ethoxylates, and copolymers thereof. Examples of commercially available ionic surfactants are sulphates (anionic) or ester sulphonates, quaternary ammonium salts (cationic) and fatty acids; imidazoquinoline derivatives such as imiquimods; synthetic derivatives of dsRNA such as Poly-IC and Poly-ICLC; trehalose dimycolate (CAF01); peptidoglycan, chitosan and combinations thereof. Organic adjuvants also include microorganism-derived adjuvants such as cholera toxin, cholera toxin B subunit, CpG DNA vaccine adjuvant, mutant E. coli labile toxin—LTR192G, monophosphoryl lipid A (Salmonella minnesota lipopolysaccharide), flagellin and flagellin-Ag fusion proteins from S. typhimurium, plant viruses such as Tobacco mosaic virus (TMV), Potato virus X (PVX), Cauliflower mosaic virus (CaMV), and Bean mild mosaic virus (BMMV).

The amount of adjuvant incorporated within the second strip will depend on the particular vaccine and the disease to be treated. Generally, an amount of adjuvant in the range of about 0.001-10% by weight of the film is incorporated within a film strip.

The film matrices of each film strip may additionally include one or more physiologically acceptable excipients to optimize the film product for a particular use, and which may vary depending on the intended mode of administration of the film. For example, the film may be administered to any mucosal membrane including the oral mucosa, nasal mucosa, ocular mucosa, rectal mucosa or vaginal mucosa.

Thus, in one embodiment, the film is formulated for oral administration and includes one or more food-grade excipients, i.e. excipients acceptable for consumption or administration to the oral cavity. Examples of suitable excipients for inclusion in a film for oral administration, which are not to be construed as limiting, include flavouring agents, sweetening agents and/or colorants.

Flavouring agents may include natural or artificial flavours such as fruit flavours (e.g. raspberry, orange, apple, pomegranate, mixed berry, lemon, lime, watermelon, strawberry, blueberry, pineapple, coconut, grape, cherry, banana, peach, mango, kiwifruit, cranberry), sodium sources (e.g. sodium chloride and monosodium glutamate), high fructose corn syrup, vanilla, chocolate, unsweetened chocolate, honey, molasses, brown sugar, coffee, cocoa, mint, maple, almond, or extracts or combinations thereof. Savoury flavourings may also be used (e.g. beef, chicken, vegetable or yeast flavourings). The film may also include a flavouring agent in an amount ranging from 0.1% to 15% by wt of the film.

Non-limiting examples of sweetening agents include natural sweeteners such as, glucose, fructose, sucrose, dextrose, maltose, brown sugar, molasses, honey, maple syrup, corn syrup, high fructose corn syrup, erythritol, xylitol, sorbitol, xylitol, neotame, saccharin, maltitol, lactitol, glycerol (glycerine), glycyrrhizin, talose, isomalt, monatin, monellin, curculin, brazzein, tagatose and mannitol, artificial sweeteners such as stevia/rebaudioside, aspartame, acesulfame K, saccharin cyclamate and sucralose, and mixtures of any of these. The film may include a sweetener in an amount in the range of 0.1% to 11% by wt.

Non-limiting examples of colorants include those which are suitable for inclusion in foods. For example, commonly used colorants include FD&C blue #1, FD&C blue #2, FD&C citrus red #2, FD&C green #3, FD&C red #3, FD&C red #40, FD&C yellow #5, FD&C yellow #6 and combinations thereof.

For ocular, nasal, rectal and vaginal films, additional excipients may be included such as a lubricant.

Preservatives may be included in any film, regardless mode of administration. Non-limiting examples of food-grade preservatives include butylated hydroxyanisole, butylated hydroxytoluene, ethylenediaminetetraacetic acid, nitrates (e.g. sodium nitrate), sulfites (sodium bisulfite), benzoates (sodium benzoate), sorbates (e.g. sodium sorbate), sodium chloride, glycerin or benzyl alcohol. Preservatives useful for other administrable forms are known in the art. For ocular and other administrable forms, benzalkonium chloride (BAK), a quaternary ammonium surfactant or a boric acid-based ionic buffer may be used.

The film may also include a pH modifier. pH modifiers suitable for oral administration, and for other administrable forms, include, for example, citric acid, phosphoric acid, malic acid, ascorbic acid, or salts thereof, ammonium carbonate, ammonium phosphate, calcium carbonate, sodium hydroxide, sodium sulfate and glutathione.

The film may additionally contain a mucosal permeation or absorption enhancer, e.g. sublingual and/or buccal permeation enhancer, to aid in delivery of the vaccine and adjuvant. An absorption enhancer in an amount in the range of about 0.01 to 5% by wt of the film may be included in the film. Examples of absorption enhancers that may be used include, but are not limited to, 23-lauryl ether, aprotinin, azone, taurine, dextran sulfate, cetylpyridinium chloride, lauric acid, menthol, xylitol, oleic acid, polyoxyethylene, polysorbate 80, salts of EDTA, deoxycholate, glycocholate, lauryl sulfate, glycodeoxycholate, salicylate, taurocholate and dodecyl sulphate (e.g. sodium salts), palmitoyl carnitine, Capsicum extract, liposome miscel and alkyl glycosides.

An anti-oxidant may also be included in the film to prevent or reduce oxidation of the active ingredients in the film. Examples of suitable anti-oxidants include, but are not limited to, sodium thiosulfate, ascorbic acid, fatty acid esters of ascorbic acid, butylated hydroxyanisole, propyl gallate, erythorbic acid, sodium erythorbate, citric acid, tocopherols such as alpha-tocopherol, and alkyl-substituted paraben or salts thereof such as ethyl paraben, methyl paraben, propyl paraben or sodium salts thereof. Antioxidant may be included in the present film in an amount suitable to reduce or prevent oxidation, for example, an amount in the range of about 0.01 to 2.5% by wt of the film.

The film may also include one or more additional active ingredients in an amount in the range of about 0.01% to 15% by wt, such as nutraceutical, pharmaceutical or therapeutic agents, including, but not limited to, botanical and/or herbal extracts, vitamins and minerals, pain medication (such as acetaminophen, non-steroidal anti-inflammatory agents (NSAIDS) such as aspirin, ibuprofen and diclofenac, anti-bacterial agents, decongestants, antihistamines, gastrointestinal treatments, and mixtures thereof. Such additional active ingredients may be combined within a strip comprising the vaccine or the adjuvant, or may be incorporated within one or more additional film strips to be laterally adjoined to the first and second film strips. As set out above, such additional film strips may each comprise a distinct film matrix, or may comprise the same film matrix as in any other strip within the film product. Generally, the film matrix will be selected based on the active ingredient(s) to be incorporated within a film strip, and the desired release profile of the active ingredient, as well as requirement for optional ingredients such as flavourants and the like.

Generally, the present films are made by blending the dry ingredients (e.g. film-forming agents) together and mixing with liquid ingredients including water, other solvents and/or an aqueous phase including water-soluble ingredients, to form a slurry. The active ingredient will be combined appropriately based on its form, e.g. dry or liquid form. Solvents are selected based on the ingredients of each film strip and may vary between film strips. The film is then extruded/cast and coated onto a moving belt or drum for drying/cutting/rolling. The drying phase may include, for example, air drying, baking, vacuum drying, microwave drying, radio-frequency drying or dehydrating with circulating warm air or other gases such as nitrogen, argon or carbon dioxide. Temperature for film extrusion is in the range of about 2-50° C. to produce a film having a moisture level in the range of about 8.5-25%, preferably about 9%. Drying conditions include a temperature of about 15-105° C. and a relative humidity of about 10-60%.

As one of skill in the art will appreciate, slot extrusion techniques are useful to achieve uniform extrusion of the film, and may also be adapted to extrude different films adjacent to one another to yield a film comprising two or more distinct adjacent conjoined strips in a single layer. In one embodiment, two or more different films are extruded in a side-by-side arrangement to yield a single layer film comprising two or more strips joined along the length of a bordering edge as shown in the Figures. In this regard, multiple extruders and specially constructed dies may be useful as described in U.S. 2004/0096569. Generally, a film-forming slurry (or slurries) is fed into a hopper (or hoppers) that channel the slurry (or slurries) for extrusion through a selected arrangement of extruder(s) and dies to achieve the desired conjoined strip-containing film.

In one embodiment, a slurry for each strip of the film may be prepared as follows. The dry ingredients (such as film-forming agents, plasticizer and active ingredients) are blended together, combined with a solvent such as potable water that has a total dissolved solid content of lower than 300 parts per million, and mixed with a high shear mixer (at about 45-55 rpm) until blended (for 10-60 minutes) at a temperature of about 65-80° C. Liquid ingredients are then added (such as surfactant/emulsifier and flavor agent) and high shear mixing is continued for 10-60 minutes at about 45-55 rpm to form a homogeneous blend. Once mixing is complete, the resulting slurry is allowed to rest for a period of time such as about two hours. Following resting, the slurry is then extruded/cast onto a moving belt, drum or mylar web which passes through a series of heating/drying (about 15-105° C.) and cooling cycles to result in a film with a 8.5-25% homogeneous moisture level, preferably about 9%, and a thickness of about 0.001″ to 0.020″ (e.g. 25 to 635 microns).

The present film may be provided with a coating of a buccal and/or sublingual mucosal absorption enhancer. A buccal and/or sublingual mucosal absorption enhancer is a compound that functions to increase absorption of the film and its content by stimulating buccal and/or sublingual mucosal absorption. Examples of suitable such absorption enhancers include, but are not limited to, 23-laurylether, aprotinin, azone, taurine, dextran sulfate, cetylpyridinium chloride, lauric acid, menthol, xylitol, oleic acid, polyoxyethylene, polysorbate 80, salts of EDTA, deoxycholate, glycocholate, lauryl sulfate, glycodeoxycholate, salicylate, taurocholate and dodecylsulphate (e.g.sodium salts), palmitoylcarnitine and alkylglycosides. For application to a film, the absorption enhancer may be encapsulated in a charged powder of maltodextrin, inulin, u-tocopherol, acacia gum, or a mixture thereof in an amount in the range of about 0.001-5% by wt. The powder is charged by exposure to a high voltage low amperage charge (e.g.60 kV to 100 kV/25 to 30 microamperes (μA)). The buccal/sublingual absorption enhancer is applied in the charged powder to both sides of the film following its manufacture at a thickness in the range of about 25-200 microns and compressed into the film, for example, by passage between two heated rollers (e.g. heated to about 50 to 98° C.).

The present film may also be provided with an anti-microbial coating, e.g. anti-bacterial, anti-fungal, anti-parasitic coating. The coating may be applied to the film in a manner similar to a buccal/sublingual absorption enhancer coating. Examples of anti-microbial agents that may be coated on the film include, but are not limited to, acetic acid, lactic acid, propionic acid, sorbic acid and benzoic acid. The amount of anti-microbial in the powdered coating is in the range of about 0.001-5% by wt, which is applied to the film at a thickness in the range of about 25-200 microns. The provision of a film with an anti-microbial coating prevents undesirable infection by a user. Since the present films are generally contained in a case, in order to consume the film, it must be removed from the case using fingers, which are not necessarily clean at all times, and then placed in the mouth, providing the opportunity for infection to a user. In some instances, the terpene within the film may function as both the active ingredient and protective anti-microbial agent.

The present films provide several advantages. A significant advantage of the present single layer films, comprising at least two distinct film strips which are laterally adjoined following the separate preparation of the film matrix for each, is the ability of the film to incorporate for delivery vaccine and adjuvant components in separate strips which avoid pre-mixing of these components and, thus, prevents or minimizes any undesired and/or premature interaction between these components prior to administration of the film. The provision of a single layer film with such laterally adjoined strips not only circumvents undesired interactions between vaccine and adjuvant, but also prevents the disadvantages associated with multi-layered films, i.e. films layered one on top of another, face to face (as opposed to edge to edge). While a multi-layered film may provide distinct layers such that a component in one layer is separated from a component in another layer, each layer of such a multi-layer laminate film product has a significant area of contact with adjacent film layers which may result in detrimental effects to the final film product. For example, rehydration and/or leaching between film layers in contact will generally occur which may result in degradation of components, may reduce efficacy of active ingredients (e.g. vaccine or adjuvant) and/or introduce complications in the manufacture of the film due to altered consistency of film matrices.

The present mucosal films also advantageously permit absorption of active ingredient (e.g vaccine/adjuvant) through mucosal membranes and avoid delivery via the gastrointestinal tract which subjects the drug to degradation from stomach acid, bile, digestive enzymes, and other effects including first-pass hepatic (i.e. liver) metabolism. As a result, the films are much more effective than other oral alternatives.

The present mucosal films also provide a suitable alternative to patients with dysphagia (i.e. swallowing disorders) and to patients suffering from nausea.

The present mucosal films are useful in a method of vaccinating a mammal. The term “mammal” is used herein to encompass both human and non-human mammals such as domestic animals, livestock and wild animals. The method comprises the step of administering the mucosal film to a mucosal membrane of the mammal, including a mucosal membrane in the oral cavity or nasal cavity, an ocular mucosal membrane, a rectal mucosal membrane or a vaginal mucosal membrane. On administration, the present film dissolves in the aqueous environment of the mucosa, and on dissolution, the vaccine and adjuvant are rapidly and effectively absorbed into the bloodstream to provide an effective plasma concentration that is at least comparable to the plasma concentration achieved by intravenous injection.

Embodiments of the invention are described in the following specific examples which are not to be construed as limiting.

### Example 1—An Edible Film Comprising Vaccine and Adjuvant is Separate Adjoined Strips

An mucosal single layer film having two dissolving adjoined strips was prepared. A first strip on the film contained an epitope-based vaccine, and a second strip of the film contained an adjuvant.

A film matrix was prepared by blending the following dry ingredients: Sorbitol (7.3 g/16.73 wt %), sodium alginate (12 g/27.5 wt %) and pullulan (19 g/43.55 wt %). Potable water (100 g) was added to the dry ingredients at 68° C. and mixed with a high shear mixer for 30 min to form a slurry matrix. Glycerin (1.45 g/3.32 wt %) was added to the slurry which was continuously blended at 50 rpm for 25 min. Polio vaccine was also added to the slurry in an amount of 2.5 g/6 wt %. Once blended, the slurry was allowed to rest for 2 hours.

Another film matrix was prepared by blending the following dry ingredients: sorbitol (7.3 g/19.16 wt %), hydroxypropyl methylcellulose (12 g/31.49 wt %), carrageen (5 g/13.12 wt %) and maltodextrin (9 g/23.62 wt %). Potable water (100 g) was added to the dry ingredients at 68° C. and mixed with a high shear mixer for 30 min to form a slurry. Glycerin (1.45 g/3.81 wt %) was added to the matrix which was continuously blended at 50 rpm for 25 min. Adjuvant (aluminum phosphate) was also added to the slurry in an amount of 2.5 g/6 wt %. Once blended, the slurry was allowed to rest for 2 hours.

The resulting slurries were prepared into a film by being drawn through an extruder adapted to separately extrude the 2 film matrices as a single layered laterally adjoined strip onto a moving Mylar web which was passed through a series of heating (25° C. to 95° C.) and cooling cycles to obtain a film having a 9% homogeneous moisture level, comprised of single layered laterally adjoined film with two distinct strips comprising different film matrices, with one strip matrix containing the vaccine and the second containing the adjuvant in side by side strips.

This film was tested and found to be effective, maintaining the vaccine and adjuvant separate, to prevent undesirable premature interaction.

1. A film for administration to a mucosal membrane comprising:
i) a first film strip comprising a vaccine in a first film matrix comprising at least one film-forming agent, and one or more of a surfactant, emulsifier and/or a plasticizer, and
ii) a second film strip comprising an adjuvant in a second film matrix comprising at least one film-forming agent, and one or more of a surfactant, emulsifier and/or a plasticizer,
wherein said first and second strips are laterally adjoined along an edge of the first and second strips.

2. The film of claim 1, which is formulated for administration to an oral, nasal, ocular, rectal or vaginal mucosal membrane.

3. The film of claim 1, formulated for oral administration.

4. The film of claim 1, wherein the vaccine is selected from the group consisting of live-attenuated viral vaccine, inactivated viral vaccine, a subunit vaccine, a recombinant vaccine, a polysaccharide vaccine, a conjugate vaccine and a toxoid vaccine.

5. The film of claim 1, comprising vaccine in an amount in the range of about 0.001% to 30% by wt of the film.

6. The film of claim 1, comprising vaccine in an amount in the range of about 10% to 30% by wt of the film.

7. The film of claim 1, wherein the adjuvant is an inorganic entity.

8. The film of claim 7, wherein the adjuvant is an aluminum-based adjuvant.

9. The film of claim 8, wherein the aluminum-based adjuvant is aluminum hydroxide, aluminum phosphate, aluminum oxide or aluminum potassium sulfate.

10. The film of claim 1, wherein the adjuvant is an organic entity.

11. The film of claim 10, wherein the organic adjuvant is an oil, saponin, cytokine, surfactant or an imidazoquinoline derivative.

12. The film of claim 10, wherein the organic adjuvant is a microorganism-derived adjuvant.

13. The film of claim 12, wherein the adjuvant is selected from the group consisting of a cholera toxin, a cholera toxin B subunit, a CpG DNA vaccine adjuvant, the mutant E. coli labile toxin, LTR192G, monophosphoryl lipid A, flagellin and flagellin-Ag fusion proteins.

14. The film of claim 1, comprising adjuvant in an amount in the range of about 0.001-10% by weight of the film.

15. The film of claim 1, comprising more than two laterally adjoined film strips.

16. The film of claim 1, wherein the film matrix of each film strip is different.

17. The film of claim 1, wherein the film-forming agent of the first and second film strip is selected from the group consisting of pullulan, a cellulose-based agent, an algin-based agent, carrageenan, amylose, high amylose starch, hydroxypropylated high amylose starch, corn starch, cassava starch, glucan, dextrin, pectin, chitin, chitosan, collagen, gelatin, zein, levan, elsinan and mixtures thereof.

18. The film of claim 1, wherein the first and second film strip comprises one or more secondary film forming agents selected from the group consisting of polyethylene glycol, polypropylene glycol, polylactic acid, sodium methacrylic acid polymers, methacrylic acid copolymers, acrylic acid polymers, acrylic acid copolymers, carboxyvinyl polymer, polyvinylpyrrolidone (PVP), carbopol, polycarbophil, cellulose gum, xanthan gum, tragacanth gum, guar gum, acacia gum, arabic gum, karaya gum, welan gum, locust bean gum, gellan gum, casein, maize, chondruscrispus, crosspovidone (CPV), glycogen, phytoglycogen, polysaccharides, hydroxypropylate, dextrin, maltodextrin, soy protein isolate, whey protein isolate, beeswax, camauba wax, fungal exopolysaccharides, rapeseed, and mixtures thereof.

19. The film of claim 1, wherein the first and second film strip comprises a film forming agent and optionally a secondary film forming agent(s) in an amount in the range of 15 to 75 wt % of the film product.

20. The film of claim 1, wherein the first and second film strip comprises a surfactant selected from the group consisting of glycerol, glycerin, propylene glycol, sucrose fatty acid ester, sorbitan monostearate, a sorbitol-based surfactant, lecithin, modified cellulose gum (CMC), ceratonia gum (locust bean gum), sodium lauryl sulfate (SLS), or mixtures thereof

21. The film of claim 1, wherein the first and second film strip comprises a surfactant in an amount ranging from 0.5 to 15% by wt.

22. The film of claim 1, wherein the first and second film strip comprises an emulsifier selected from the group consisting of polyoxyethylene, sorbitan monostearate, lecithin, stearate salt, calcium stearoyl lactylate (CSL), glyceryl monostearate, mono propylene glycol, sorbitan monooleate (SPAN 80), a sorbitol-based emulsifier, monoglyceride, glyceroltriacetate and mixtures thereof.

23. The film of claim 1, wherein the first and second film strip comprises a plasticizer selected from the group consisting of monostearate palm oil, lethicin, sorbitan monostearate, glycerin, glycerin fatty acid ester, triacetin, pre-gelatinized starch, sorbitol, medium chain mono- or diglycerides, castor oil, malic acid, polyethylene glycol (PEG), glycerol, palm oil and mixtures thereof

24. The film of claim 1, wherein the first and second film strip comprises a plasticizer in an amount ranging from 2 to 18% by wt.

25. The film of claim 1, wherein the first film strip comprises pullulan and/or sodium alginate, and the second film strip comprises hydroxypropyl methylcellulose and/or carrageen.

26. The film of claim 25, wherein the first and second film strips additionally comprise glycerin.

27. The film of claim 1, wherein the vaccine is a polio vaccine, and the adjuvant is an aluminum-based adjuvant.

28. The film of claim 1, which is a single layer film.
